(19) United States
(12) Patent Application Publication
(10) Pub. No.: US 2009/0312752 A1
(43) Pub. Date: Dec. 17, 2009

(54) Medical Laser Vacuum Evacuator

(76) Inventor: Zoran Djordjevich, Encinitas, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/137,374

(22) Filed: Jun. 11, 2008

(51) Int. Cl. A61B 18/20

(52) U.S. Cl. 606/10

(57) ABSTRACT

A smoke evacuating apparatus including a nose component having a centrally positioned receptacle for receiving and supporting the tip of a medical laser device and including a chamber through which smoke generated by the laser may be evacuated, a body component removably attachable to the nose component and including a centrally mounted tube for receiving and supporting the laser and an internal smoke evacuating passageway for receiving smoke from the chamber and a vacuum hose providing a second internal passageway surrounding the laser for communicating with the first passageway and receiving smoke therefrom, the hose including a first tube for sealably mounting a rearward portion of the laser and a second tube disposed at an angle to the first tube for attachment to a vacuum source.

### FIELD

The subject disclosure relates generally to medical devices and more particularly to a vacuum evacuator for removing smoke generated by a medical laser.

### BACKGROUND

Conventional medical lasers are used, for example, to remove tissue and/or stop bleeding during various procedures. The operation of such a laser produces smoke, which obscures the view of the doctor or other individual using the laser.

### SUMMARY

The following is a summary description of an illustrative embodiment of the invention. It is provided as a preface to assist those skilled in the art to more rapidly assimilate the detailed design discussion which ensues and is not intended in any way to limit the scope of the claims which are appended hereto in order to particularly point out the invention.

An illustrative embodiment of a smoke evacuating apparatus for use with a medical laser includes a nose component, a body component and a vacuum hose. The nose component has a centrally positioned receptacle for receiving and supporting the tip of a medical laser device and includes a chamber through which smoke generated by the tip of the laser may be evacuated. The nose component attaches to the body component, which includes an internally mounted tube for receiving and further supporting the laser device and provides an internal smoke evacuating passageway for receiving smoke from the chamber in the nose component. The tubular vacuum hose provides a second internal passageway surrounding the laser. The second passageway communicates with the first passageway to receive and evacuate smoke from the body component. The tubular vacuum hose further includes a first tube for sealably receiving and supporting the laser and a second tube disposed at an angle to the first tube for attachment to a vacuum source.

### DESCRIPTION OF DRAWINGS

The features and objects of the present disclosure will become more apparent with reference to the following description taken in conjunction with the accompanying drawings wherein like reference numerals denote like elements and in which:

FIG. 1 is an exploded perspective view depicting an illustrative embodiment of the invention;

FIG. 2 is a perspective view of a nose component of the illustrative embodiment;

FIG. 3 is a front view of the nose component of FIG. 2;

FIG. 4 is a perspective view of a body component of the illustrative embodiment;

FIG. 5 is a side view of the body component of FIG. 4;

FIG. 6 is a front view of a body component of FIG. 4;

FIG. 7 is a cross-sectional view illustrating the front portion of the illustrative embodiment in assembled state encasing a medical laser instrument;

FIG. 8 is a cross-sectional view of the rear portion of the illustrative embodiment in the assembled state.

FIG. 9 is a perspective view of the illustrative embodiment in the assembled state;

FIG. 10 is a disassembled perspective view illustrating an alternative embodiment; and

FIG. 11 is a perspective view illustrating an optional smoke filter component.

### DETAILED DESCRIPTION

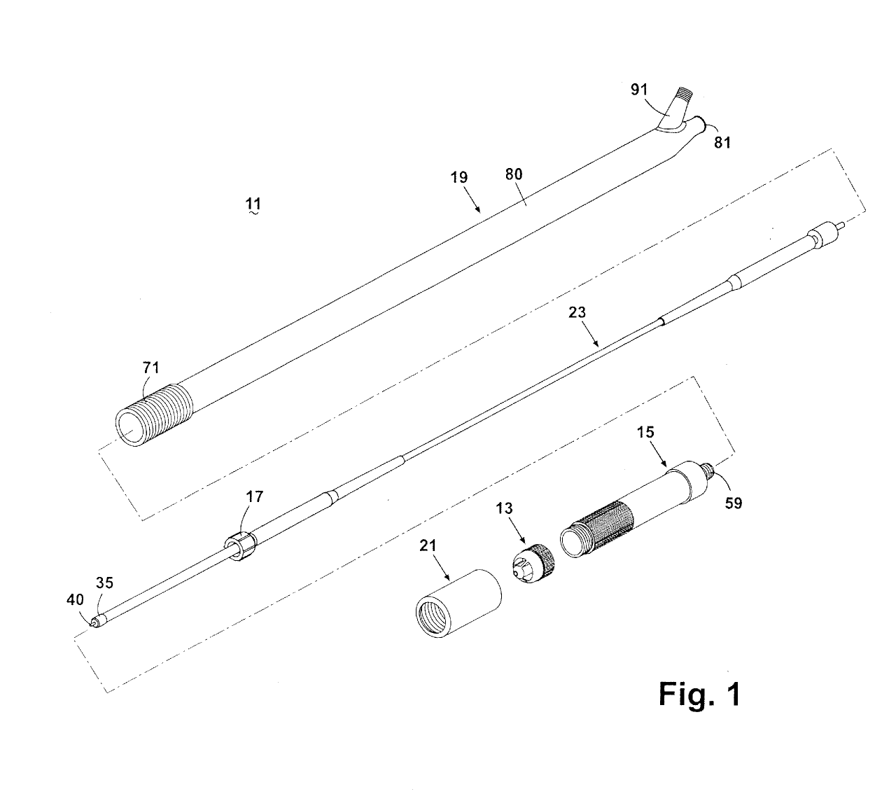

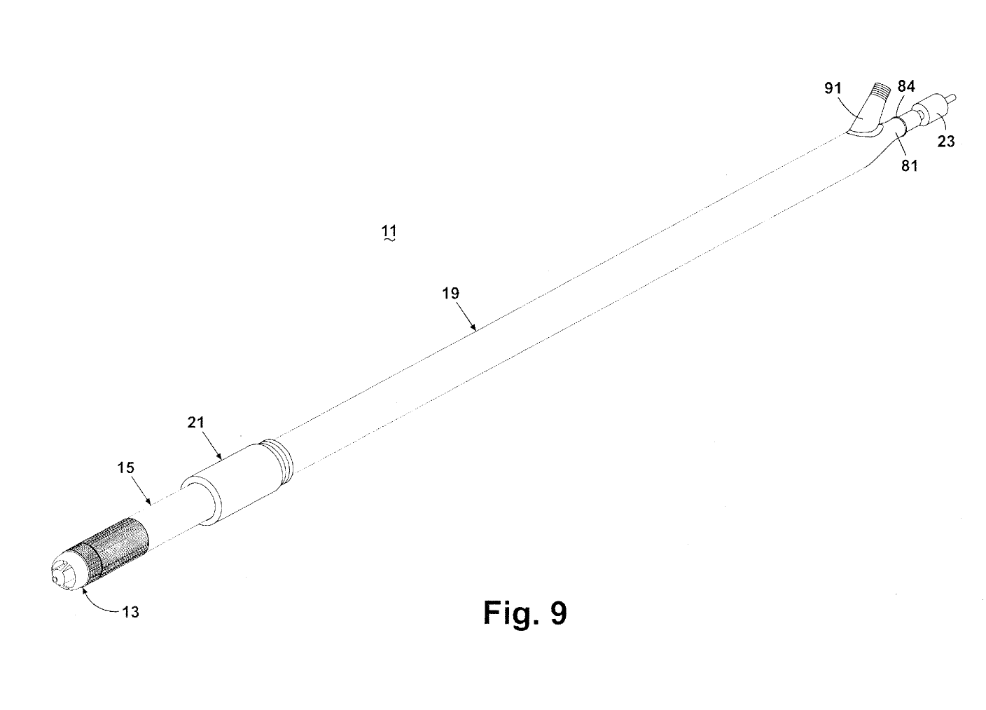

FIGS. 1-9 illustrate a medical laser smoke evacuator 11 according to an illustrative embodiment. The evacuator 11 includes a nose 13, a body 15, a vacuum hose component 19, and a coupler 21. These components 13, 15, 19, 21 form a housing into which a conventional medical laser 23 is inserted and encased. An example of such a laser 23 is a Luxar Faser PN LXFR-LF1.0 00867-03.

During use, such a conventional medical laser 23 may be employed to, for example, remove tissue and/or stop bleeding. Such operations produce smoke, which obscures the view of the doctor or other individual using the laser. Such smoke is removed by the illustrative embodiment, providing better vision for the doctor.

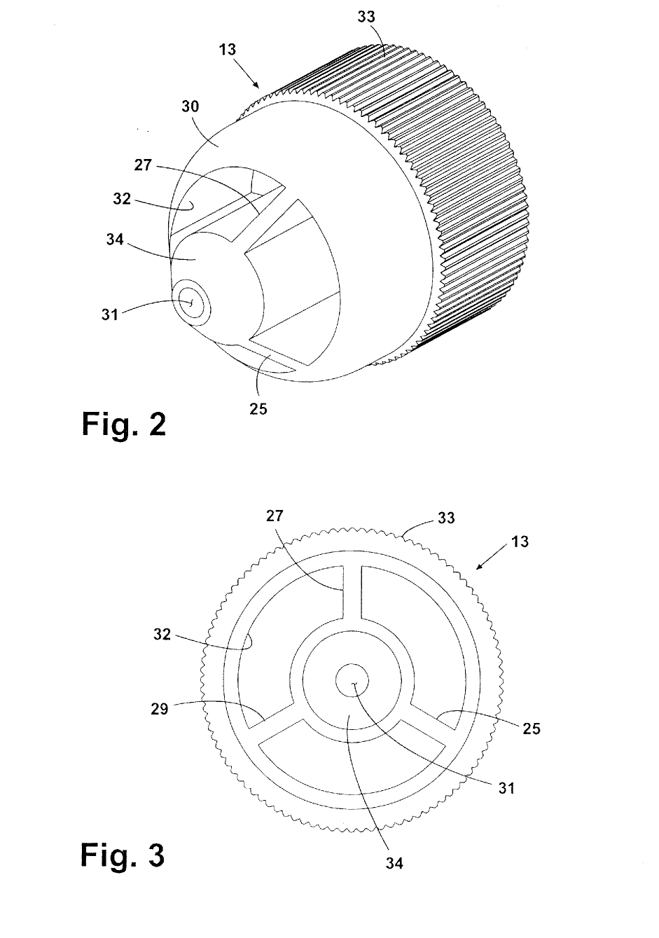

The nose component 13 of the illustrative embodiment is shown in more detail in FIGS. 2 and 3. The outer surface of the nose 13 includes a front portion 30 and a cylindrical rear portion 33, which is knurled for better grip. The interior of the nose 13 includes an interior cylindrical surface 32 from which extend three radially disposed ribs 25, 27, 29, each separated from the other, for example, by 120 degrees.

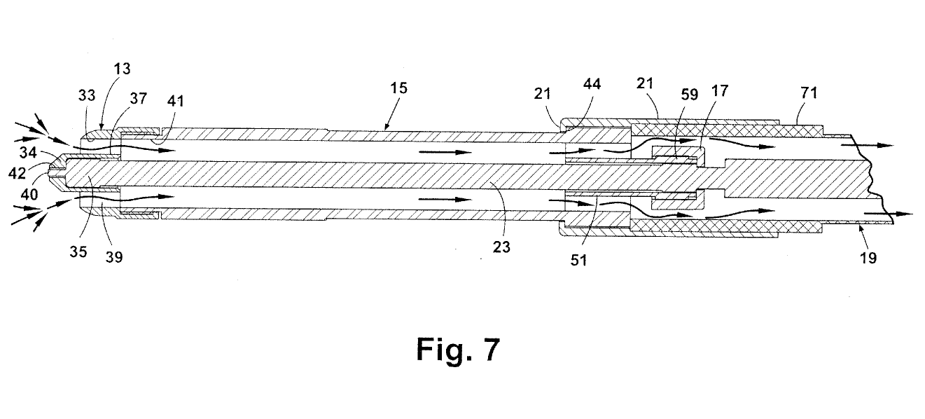

The three radial ribs 25, 27, 29 support a cylindrical tip mounting receptacle 34 and concentrically position that receptacle 34 within the inner cylindrical surface 32. The receptacle 34 is shaped and dimensioned to receive and position the end 35 of the laser 23, for example, as illustrated in FIG. 7. The receptacle 34 further includes an opening 31, which passes the light-emitting tip 40 of the laser 23, which tip, for example, may be approximately 10 mm long and 2-3 mm wide.

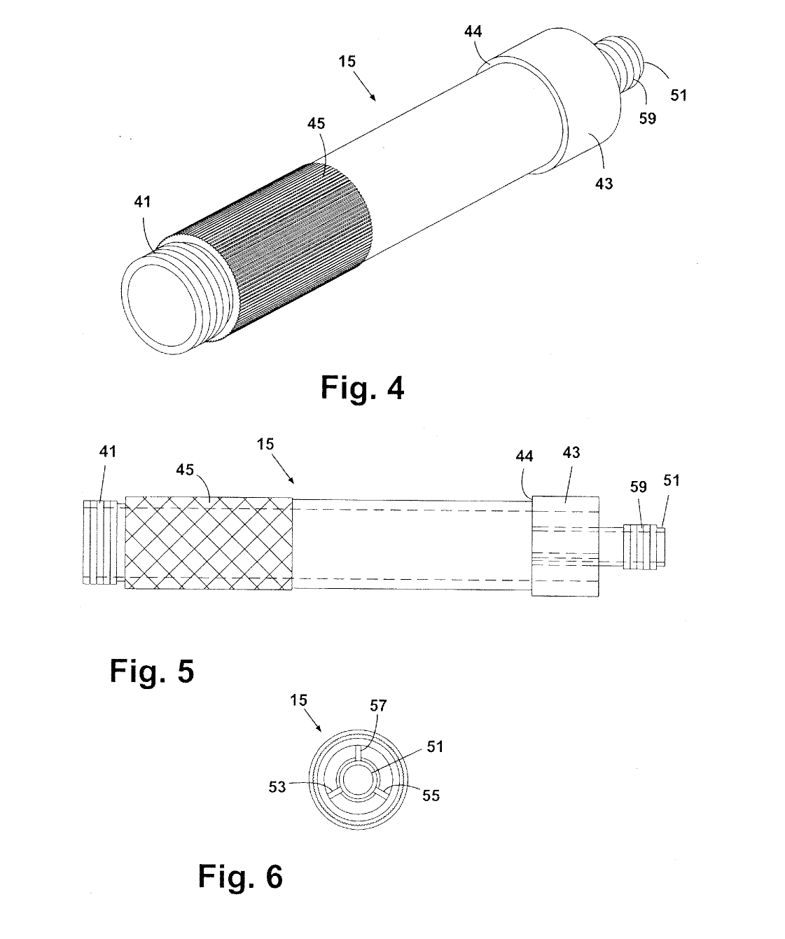

The body component of the illustrative embodiment 15 is shown in more detail in FIGS. 4-6. As may be seen, the body 15 is a generally cylindrical component with one threaded end 41 and one unthreaded end 43. A knurled surface 45 is provided adjacent the threaded end 41. The knurled surface 45 mates with the knurled surface 33 of the nose 13 when the end 41 of the body 15 is threaded into the mating threads of the nose 13, as shown in FIG. 7. The unthreaded end 43 provides an annular edge 41, which abuts the depending lip 21 of the coupler 21 in the assembled state shown in FIG. 7.

The body 15 further includes a tube 51, concentrically and centrally positioned within the generally cylindrical interior of the body 15 by three ribs 53, 55, 57, each disposed 120° from the other. The tube 51 includes a threaded end 59 onto which nut 17 of the laser 23 threads. Thus, the tube 51 receives, passes, and supports the laser 23, as shown in FIG. 7.

As seen in FIGS. 1 and 7, the coupler 21 is generally cylindrical with a depending annular lip 21 formed at one end thereof. The coupler is further internally threaded to receive and mate with the threads 71 at the end of the vacuum hose 19.

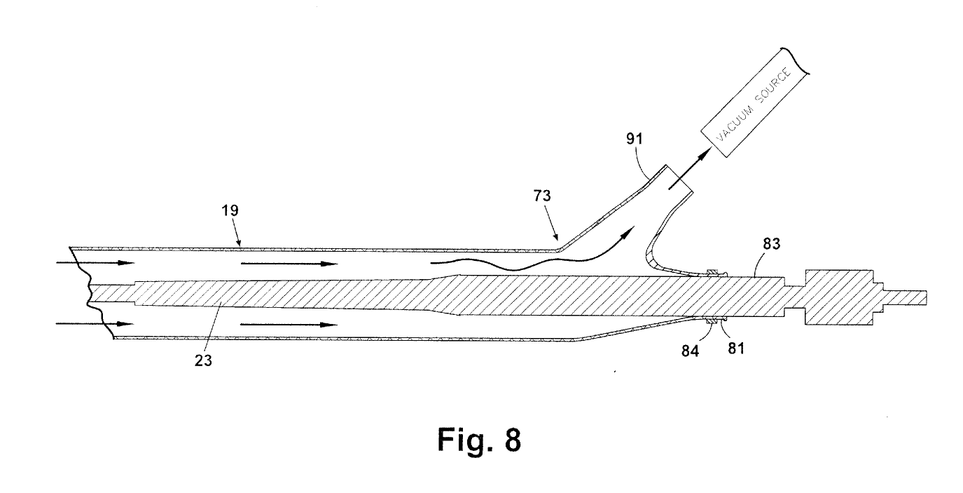

The opposite end 73 of the vacuum hose 19 comprises a tube 81 of circular cross-section, which is shaped and dimensioned to seal against the outer surface of a rear portion 83 of the laser 23, as shown in FIG. 8. The opposite end 71 further includes a tubular passageway 91 disposed at an angle to the body 80 of the hose 19. The passageway 91 attaches to a vacuum hose or other device which applies smoke-evacuating suction through the tubular hose 19 and body 15 to the nose 13 of the smoke evacuator 11.

As may be seen in FIG. 7, the end 35 of the laser 23 fits snugly and conformingly within the generally cylindrical interior of the receptacle 34. The cylindrical exterior surface 37 of the receptacle 34 and the interior cylindrical surface 33 of the nose 13 define a chamber 39 through which smoke generated by action of the laser 23 is evacuated in the direction of the arrows. In an illustrative embodiment, the inside diameter “d₁” of chamber 39 may be, for example, 6.8 millimeters (mm), while the diameter “d₂” of the outer cylinder 33 may be 12.5 mm in an illustrative embodiment. Additionally, in the illustrative embodiment, the tip 40 at the end 35 of the laser 23 is substantially flush with the end 42 of the receptacle 37.

To assemble the device 11, the laser 23 is passed through the tube 51 of the body component 15, and the nut 17 of the laser 23 is then threaded onto the threaded end 59 of the tube 51. The nose 13 is then threaded onto the body 15. Next, the coupler 21 is slid over the nose 13 and body 15. The vacuum hose 19 is then slid over the laser 23, and the coupler 21 is threaded onto the threaded end 71 of the hose 19. A clamp 84 (FIG. 8) is then applied about the end 81 to seal the tube 81 against the laser 23.

The illustrated vacuum hand piece will efficiently remove smoke during laser surgical procedures with less power since the smoke intake is in close proximity to smoke origination. Smoke will not obscure the surgeon's view and the vacuum device will maintain a healthy “smokeless” working environment. Employing the hose 19 to encase the laser 23 minimizes space occupied by the vacuum machine and is more convenient for the surgeon. The illustrative embodiment also eliminates the need for a second assistant, or “hose holder” sometimes employed in the past. The components 13, 15 and 21 may be easily disassembled for sterilization and provide a larger sterile gripping surface for the surgeon's hands.

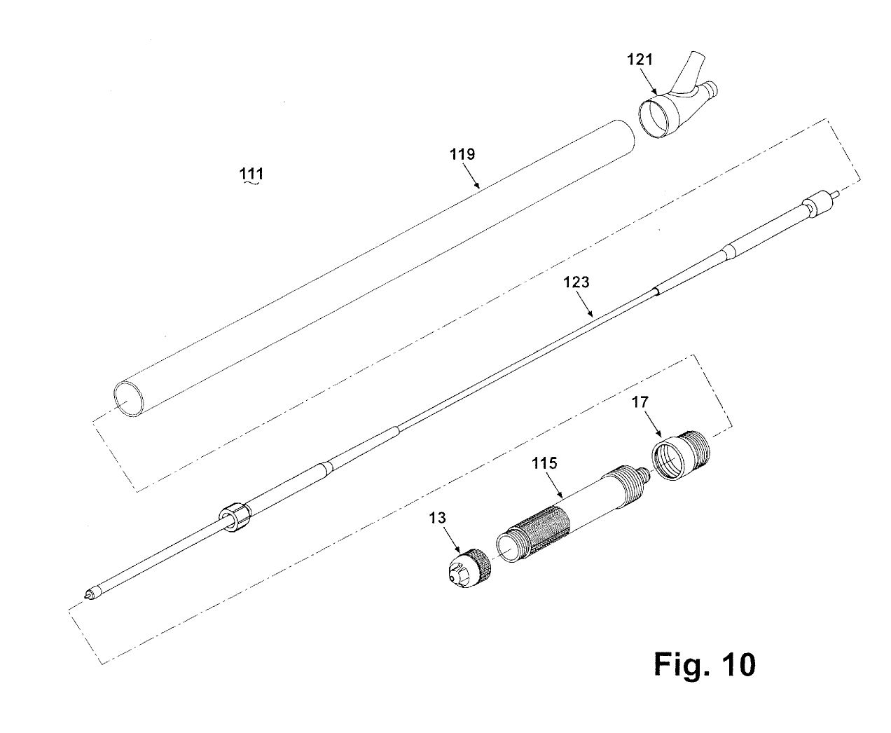

FIG. 10 illustrates an alternative embodiment 111 wherein an adapter component 117 threads onto a threaded end of a body component 115 and press-fits into or otherwise attaches to a hose component 119. The embodiment of FIG. 10 further includes a separate Y adaptor 121, which attaches to the hose 119 for receiving the laser 123 and a vacuum hose attachment.

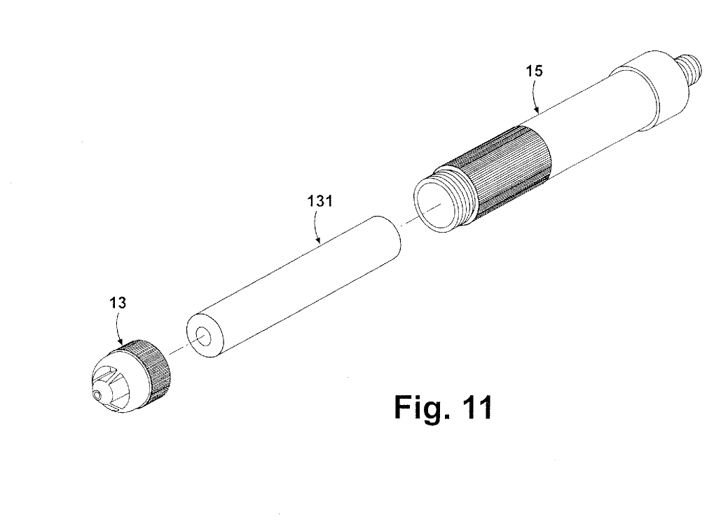

Optionally, as shown in FIG. 11, a cylindrical smoke filter 131 of annular cross-section can be placed concentrically within the housing component 15 and dimensioned to be captured and held in place by the nose component 13. Such a filter may be formed, for example, from a porous or perforated plastic sheet rolled or molded into the form of a cylinder, and may be designed to remove larger smoke particles.

Those skilled in the art will appreciate that various adaptations and modifications of the just described embodiment can be configured without departing from the scope and spirit of the invention. Therefore, it is to be understood that, within the scope of the appended claims, the invention may be practiced other than as specifically described herein.

1. A smoke evacuating apparatus comprising:
a nose component including a centrally positioned receptacle for receiving and supporting a tip of a medical laser device and including a chamber through which smoke generated by operation of said laser may be evacuated;
a body component removably attachable to said nose component and including a centrally mounted tube for receiving, passing and supporting said laser and including a first internal smoke evacuating passageway for receiving and evacuating smoke from said chamber; and
a tubular vacuum hose providing a second internal passageway surrounding said laser for communicating with said first passageway and receiving and evacuating smoke therefrom, said hose including a first tubular portion for receiving and supporting a portion of said laser and a second tubular portion disposed at an angle to said first tubular portion for attachment to a vacuum source, the first tubular portion being sealed about said portion of said laser.

2. The apparatus of claim 2 further including a coupler for attaching said body component to said tubular vacuum hose.

3. The apparatus of claim 1 further including a vacuum source connected to said second tubular portion for applying a vacuum thereto.

4. The apparatus of claim 1 further including a cylindrical filter positioned within said body component.
